(19) United States
(12) Patent Application Publication
(10) Pub. No.: US 2010/0173947 A1
(43) Pub. Date: Jul. 8, 2010

(54) New Indications for Direct Thrombin Inhibitors

(75) Inventors: Andreas Clemens, Wiesbaden (DE); Paul A. Reilly, Ridgefield, CT (US)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 12/728,732

(22) Filed: Mar. 22, 2010

Related U.S. Application Data
(63) Continuation of application No. 11/778,748, filed on Jul. 17, 2007, now abandoned.

(30) Foreign Application Priority Data
Jul. 17, 2006 (EP) 06117345
Feb. 15, 2007 (EP) 07102513

(51) Int. Cl. A61K 31/4439; A61K 31/4709; A61K 38/17; A61K 31/397; A61K 31/4184; A61P 35/00; A61P 25/28; A61P 29/00; A61P 13/12; A61P 7/02

(52) U.S. Cl. 514/338; 514/314; 514/12; 514/210.17; 514/394

(57) ABSTRACT

The invention relates to new indications for direct thrombin inhibitors such as dabigatran etexilate in the CNS and other fields.

### RELATED APPLICATIONS

This application claims priority to co-pending U.S. application Ser. No. 11/778,748, filed Jul. 17, 2007, which claims priority to European Patent Application Nos. 06 117 345 and 07 102 513, filed Jul. 17, 2006 and Feb. 15, 2007, respectively, the contents of which are incorporated herein by reference in their entirety.

The present invention relates to novel indications for direct thrombin inhibitors (DTI), processes for preparing pharmaceutical compositions for treating said diseases and methods of treating them.

### DETAILED DESCRIPTION OF THE INVENTION

Direct thrombin inhibitors according to the invention include
(1) 1-methyl-2-(4-amidinophenylaminomethyl)-benzimidazol-5-yl-carboxylic acid-(N-2-pyridyl-N-2-hydroxycarbonylethyl)-amide known as dabigatran having the structure

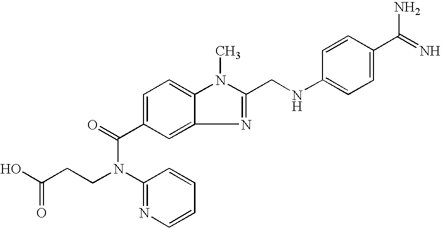

(2) ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate known as dabigatran etexilate having the following structure

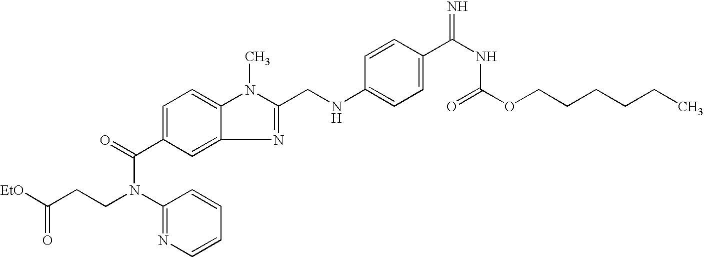

(3) 1-methyl-2-[4-(N-hydroxyamidino)-phenylaminomethyl]-benzimidazol-5-yl-carboxylic acid-(N-2-pyridyl-N-2-ethoxycarbonylethyl)-amide having the structure

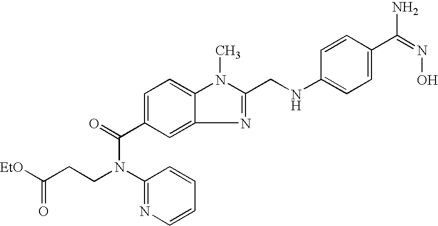

(4) melagatran (inogatran),
(5) ximelagatran,
(6) hirudin,
(7) hirolog and
(8) argatroban,
   optionally in the form of tautomers, racemates, enantiomers, diastereomers, pharmacologically acceptable acid addition salts, solvates, hydrates or prodrugs thereof.

Preferred direct thrombin inhibitors are dabigatran, dabigatran etexilate and 1-methyl-2-[4-(N-hydroxyamidino)-phenylaminomethyl]-benzimidazol-5-yl-carboxylic acid-(N-2-pyridyl-N-2-ethoxycarbonylethyl)-amide, and the tautomers, racemates, enantiomers, diastereomers, pharmacologically acceptable acid addition salts, solvates, hydrates and prodrugs thereof.

More preferred are dabigatran and dabigatran etexilate, and the tautomers, racemates, enantiomers, diastereomers, pharmacologically acceptable acid addition salts, solvates, hydrates and prodrugs thereof.

Most preferred is dabigatran etexilate, and the tautomers, racemates, enantiomers, diastereomers, pharmacologically acceptable acid addition salts, solvates, hydrates and prodrugs thereof, particularly its acid addition salt with methanesulfonic acid.

All active components should be used in effective amounts.

The active compounds (1) to (3) are disclosed in the prior art, e.g. in WO 98/37075 and WO 04/014894. The acid addition salt of dabigatran etexilate with methanesulfonic acid is described in WO 03/074056. Additional salts of dabigatran etexilate are mentioned in the experimental part. Specific polymorphs and a hemihydrate of acid addition salt of dabigatran etexilate with methanesulfonic acid is described in WO 2005/028468. Examples for pharmaceutical composition containing dabigatran etexilate are disclosed in WO 03/074056, WO 2005/018615 and WO 2005/023249.

Prodrugs of the drugs mentioned above are such derivatives containing one or more groups capable of being cleaved in vivo, particularly a group which can be converted in vivo into a carboxy group or/and a group capable of being cleaved in vivo from an imino or amino group. Compounds containing two groups capable of being cleaved in vivo are so-called double prodrugs. Groups which can be converted in vivo into a carboxy group and groups capable of being cleaved in vivo from an imino or amino group are disclosed e.g. in WO 98/37075, being herewith incorporated by reference, as well as in other WO publications cited hereinbefore in connection with specific antithrombotics.

It is understood that the direct thrombin inhibitor according to the invention may be used in a form selected from tautomers, optical isomers, enantiomers, racemates, diastereomers, pharmacologically acceptable acid addition salts, solvates or hydrates, as far as such forms exist, depending on the individual compound. If multiple enantiomers exist, the use in form of a substantially pure enantiomer is preferred.

Pharmacological acceptable acid addition salts of the direct thrombin inhibitors listed above comprise salts selected from the group consisting of the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrolactate, hydrooxalate, hydrosuccinate, hydrobenzoate and hydro-p-toluolsulphonate, preferably hydrochloride, hydrobromide, hydrosulphate, hydrophosphate, hydromaleate, hydrofumarate and hydromethansulphonate. Some of the direct thrombin inhibitors may add more than one equivalent acid, e.g. two equivalents. The salts of hydrochloric acid, methanesulfonic acid, maleic acid, benzoic acid and acetic acid are especially preferred.

A preferred embodiment are the salts of dabigatran etexilate with hydrochloric acid, maleic acid, tartaric acid, salicylic acid, citric acid, methanesulfonic acid and malonic acid, the enantiomers, mixtures and hydrates thereof. Particularly preferred are tartaric acid, salicylic acid, methanesulfonic acid and citric acid as well as the enantiomers, mixtures and hydrates thereof. The most preferred salt of is the methanesulfonic acid addition salt of dabigatran etexilate.

The following terms are used synonymously:

salt with hydrochloric acid-hydrochloride
salt with maleic acid-maleate
salt with tartaric acid-tartrate
salt with salicylic acid-salicylate
salt with citric acid-citrate
salt with malonic acid-malonate
salt with methanesulfonic acid-methanesulfonate

Any reference to a direct thrombin inhibitor within the scope of the present invention should be understood as a reference to any specific direct thrombin inhibitor selected from compounds (1) to (8) mentioned hereinbefore.

A preferred embodiment of the invention relates to new indications of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, the salts, the enantiomers, the mixtures and the hydrates thereof. This active substance with the chemical formula

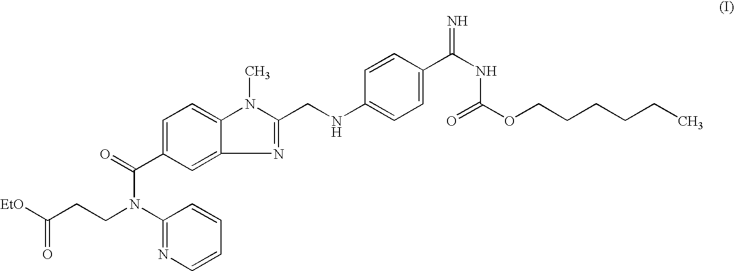

is already known from WO 98/37075, wherein compounds with a thrombin-inhibiting and thrombin time-prolonging activity are disclosed, under the name 1-methyl-2-[N-[4-(N-n-hexyloxycarbonylamidino)phenyl]-amino-methyl]-benzimidazol-5-yl-carboxylic acid-N-(2-pyridyl)-N-(2-ethoxycarbonylethyl)-amide. The compound of formula I is a double prodrug of the compound

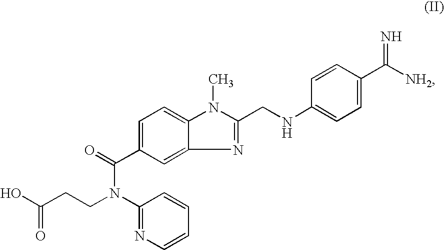

i.e. the compound of formula I is first converted into the actual effective compound, namely the compound of formula II, in the body. The main type of indication for the compound of chemical formula I is the post-operative prophylaxis of deep vein thrombosis and the prevention of strokes.

Surprisingly, the direct thrombin inhibitors like e.g. dabigatran etexilate cannot only be used effectively for the post-operative prophylaxis of deep vein thrombosis and the prevention of strokes, but are also suitable for the prevention and/or treatment of other diseases.

Accordingly, the invention is related to the use of a compound, optionally in the form of tautomers, racemates, enantiomers, diastereomers, pharmacologically acceptable acid addition salts, solvates, hydrates or prodrugs thereof, selected from the group consisting of dabigatran, dabigatran etexilate, 1-methyl-2-[4-(N-hydroxyamidino)-phenylaminomethyl]-benzimidazol-5-yl-carboxylic acid-(N-2-pyridyl-N-2-ethoxycarbonylethyl)-amide, melagatran (inogatran), ximelagatran, hirudin, hirolog and argatroban for preparing a medicament for the treatment and/or prophylaxis of a disease selected from the group consisting of:
neurodegenerative disease, in particular Alzheimer disease, brain micro vessel disease,
diseases which are mediated via PAR 1 to PAR 4 receptors and oxidative stress induced by thrombin.

In another preferred embodiment the invention is related to the use of the compounds mentioned hereinbefore for preparing a medicament for the treatment and/or prophylaxis of
Haematological diseases,
heparin induced thrombocythompenia,
disseminated intravascular coagulation (DIC).

In another preferred embodiment the invention is related to the use of the compounds mentioned hereinbefore for preparing a medicament for the treatment and/or prophylaxis of a disease selected from among
thrombosis,
thrombosis in polychemotherapy in patients suffering from cancer.

In another preferred embodiment the invention is related to the use of the compounds mentioned hereinbefore for preparing a medicament for the treatment and/or prophylaxis of cancer, in particular
lung cancer,
pancreatic cancer.

In another preferred embodiment the invention is related to the use of the compounds mentioned hereinbefore for preparing a medicament for the treatment and/or prophylaxis of central vein thrombosis (CVT).

In another preferred embodiment the invention is related to the use of the compounds mentioned hereinbefore for preparing a medicament for the treatment and/or prophylaxis of HIV encephalitis in patients suffering from human immunodefience virus (HIV).

In another preferred embodiment the invention is related to the use of the compounds mentioned hereinbefore for preparing a medicament for the treatment and/or prophylaxis of rheumatoid disorders, in particular
rheumatoid arthritis and
systemic lupus erythematodes (SLE).

In another preferred embodiment the invention is related to the use of the compounds mentioned hereinbefore for preparing a medicament for the treatment and/or prophylaxis of Tinnitus Aurium.

In another preferred embodiment the invention is related to the use of the compounds mentioned hereinbefore for preparing a medicament for the treatment and/or prophylaxis of kidney disease, in particular
proteinuria (urinary albumin excretion) in patients with chronic kidney disease and
proteinuria (urinary albumin excretion) in patients with Diabetes and albuminuria.

Besides the treatment and/or prophylaxes of these diseases the thrombin inhibitors listed above are useful for the prevention and/or treatment of events provoked by the above-mentioned diseases (like VTE, PE), optimize the blood flow to organs or regions, and/or are suitable for direct treatment of the diseases.

A preferred embodiment is the use of the direct thrombin inhibitors according to the invention for the preparation of a medicament for treating or preventing VTE associated with any one of the diseases mentioned above resp. below.

Preferred indications are:
1) CNS-field
   a. neurodegenerative disease (e.g. Alzheimer disease)
   b. brain micro vessel disease
   c. diseases which are mediated via PAR 1 to PAR 4 receptors
   d. oxidative stress induced by thrombin
2) Haematology
   a. Heparin induced thrombocythompenia
   b. Patients with elevated coagulant parameters (e.g. PAI 1)
3) Cancer
   a. Treatment and/or prophylaxes and/or secondary prevention of cancer, in particular lung cancer or pancreatic cancer
   b. Prevention of thrombosis in polychemotherapy
   c. Prevention of thrombosis in cancer patients, in particular in lung cancer patients or pancreatic cancer patients
   d. Treatment of thrombosis in cancer patients, in particular in lung cancer patients or pancreatic cancer patients
   e. Mortality reduction as mono-therapy and in combination with anti-cancer agents
4) Opthamology
   a. Central vein thrombosis (CVT)
5) Human Immunodefience Virus (HIV) patients
   a. HIV encephalitis
6) Rheumatoid disorders
   a. Rheumatoid arthritis
   b. Systemic Lupus erythematodes (SLE)
7) Patients with transplantation
8) Tinnitus Aurium
9) Kidney disease
   a. Proteinuria (urinary albumin excretion) in patients with chronic kidney disease
   b. Proteinuria (urinary albumin excretion) in patients with Diabetes and albuminuria.

In another embodiment the invention relates to the use of the compounds mentioned hereinbefore for preparing a medicament for the treatment and/or prophylaxis of one or several of the diseases mentioned hereinbefore, wherein the disease is associated with VTE.

The direct thrombin inhibitor, optionally used in form of its pharmaceutically acceptable acid addition salts, may be incorporated into the conventional pharmaceutical preparation in solid, liquid or spray form. The composition may, for example, be presented in a form suitable for oral, topical, lingual, rectal, parenteral administration or for nasal inhalation: preferred forms includes for example, capsules, tablets, coated tablets, ampoules, suppositories and nasal spray.

The active ingredient may be incorporated in excipients or carriers conventionally used in pharmaceutical compositions such as, for example, talc, arabic gum, lactose, gelatine, magnesium stearate, corn starch, acqueous or non acqueous vehicles, polyvinyl pyrrolidone, semisynthetic glicerides of fatty acids, benzalconium chloride, sodium phosphate, EDTA, polysorbate 80. The compositions are advantageously formulated in dosage units, each dosage unit being adapted to supply a single dose of the active ingredient. The dosis range applicable per day is between 0.1 mg to 600 mg, preferably between 50 mg to 300 mg/day. Each dosage unit may conveniently contain from 0.1 mg to 200 mg, preferably from 50 mg to 150 mg.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g of. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g of. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

### EXAMPLES

The Examples which follow illustrate the present invention without restricting its scope:

The starting material dabigatran etexilate (ethyl 3-[(2-{[4-(amino-hexyloxy-carbonylimino-methyl)-phenylamino]-methyl}-1-methyl-1H-benzimidazole-5-carbonyl)-pyridin-2-yl-amino]-propionate) may for example be prepared as described in International Application WO 98/37075, Example 113.

### Example 1

### Hydrochloride of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

125 mg (1.59 mmol) of acetyl chloride were added to 5 ml ethanol with stirring. The solution thus obtained was then added dropwise at ambient temperature to a solution of 1.0 g (1.59 mmol) of ethyl 3-[(2-{[4-(amino-hexyloxycarbonylimino-methyl)-phenylamino]-methyl}-1-methyl-1H-benzimidazole-5-carbonylypyridin-2-yl-amino]-propionate and stirred for a further two hours. The mixture was then evaporated down completely, the residue was first of all triturated after the addition of approx. 5 ml ethyl acetate and suction filtered, then stirred overnight in approx. 10 ml acetone, suction filtered, washed with a little acetone and diethyl ether and then dried at 60° C. in vacuo.

Yield: 86% of theory

Melting point: 135° C.

### Example 2

### Citric Acid Salt of ethyl 3-[(2-{[4-(amino-hexyloxycarbonylimino-methyl)-phenyl-amino]-methyl}-1-methyl-1H-benzimidazole-5-carbonyl)-pyridin-2-yl-amino]-propionate

210 mg (1.0 mmol) of citric acid hydrate, dissolved in 10 ml ethyl acetate, were added dropwise at ambient temperature with stirring to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate in 45 ml ethyl acetate. A yellow precipitate formed. The mixture was stirred overnight, the product was then suction filtered, washed with a little ethyl acetate and diethyl ether and dried at approx. 50° C. in vacuo.

Yield: 83% of theory

Melting point: approx. 170° C. (with decomposition)

### Example 3

### Tartaric Acid Salt of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

150 mg (1.0 mmol) of L(+)-tartaric acid, dissolved in 5 ml absolute ethanol, were added dropwise at ambient temperature with stirring to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonylypyridin-2-yl-amino]-propionate in 50 ml ethyl acetate. A fine precipitate was formed. The suspension was stirred for a further two hours, then the product was suction filtered, washed with a little cold ethyl acetate and diethyl ether and dried in vacuo at approx. 50° C.

Yield: 72% of theory

Melting point: approx. 160° C. (with decomposition)

### Example 4

Malonic acid salt of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

104 mg (1.0 mmol) of malonic acid, dissolved in 10 ml ethyl acetate, were added dropwise at ambient temperature, with stirring, to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonylypyridin-2-yl-amino]-propionate in 50 ml ethyl acetate. After approx. one hour a fine precipitate formed. The suspension was stirred for a further three hours, the product was then suction filtered, washed with a little cold ethyl acetate and diethyl ether and dried in vacuo at approx. 50° C.

Yield: 79% of theory

Melting point: 100° C.

### Example 5

Maleic acid salt of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

116 mg (1.0 mmol) of maleic acid, dissolved in 10 ml ethyl acetate, were added dropwise, with stirring, at ambient temperature, to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonylypyridin-2-yl-amino]-propionate in 50 ml ethyl acetate. A precipitate formed. The suspension was stirred for a further three hours, then the product was suction filtered, washed with a little cold ethyl acetate and diethyl ether and dried in vacuo at approx. 50° C.

Yield: 93% of theory

Melting point: 120° C.

### Example 6

### Ethyl-3-[(2-{[4-(hexyloxycarbonylamino-imino-methyl)-phenylamino]-methyl}-1-methyl-1H-benzimidazole-5-carbonyl)-pyridin-2-yl-amino]-propionate salicylate

A solution of 1.38 g (10.0 mmol) of salicylic acid in 20 ml acetone was added dropwise with stirring at 35-40° C. to a solution of 6.28 g (10.0 mmol) of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base (prepared as described in WO 98/37075), in 45 ml acetone. After a few minutes the product began to crystallise out and it was diluted with 65 ml acetone. Within 30 minutes the mixture was cooled to ambient temperature, then the precipitate was suction filtered, washed with approx. 40 ml acetone and dried at 40° C. in the circulating air dryer.

Yield: 94% of theory

Melting point: 155° C.

### Example 7

Dry ampoule containing 75 mg active substance per 10 ml

| Composition: | | |
|---|---|---|
| active substance | 75.0 | mg |
| mannitol | 50.0 | mg |
| water for injections | ad 10.0 | ml |

### Preparation:

Active substance and mannitol are dissolved in water. After packaging the solution is freeze-dried. To produce the solution ready for use for injections, the product is dissolved in water.

### Example 8

Dry ampoule containing 35 mg of active substance per 2 ml

| Composition: | | |
|---|---|---|
| Active substance | 35.0 | mg |
| Mannitol | 100.0 | mg |
| water for injections | ad 2.0 | ml |

### Preparation:

Active substance and mannitol are dissolved in water. After packaging, the solution is freeze-dried.

To produce the solution ready for use for injections, the product is dissolved in water.

### Example 9

Tablet containing 50 mg of active substance

| Composition: | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

### Preparation:

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 9 mm.

### Example 10

Tablet containing 350 mg of active substance

| Composition: | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

### Preparation:

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 12 mm.

### Example 11

Capsules containing 50 mg of active substance

| Composition: | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

### Preparation:

(1) is triturated with (3). This trituration is added to the mixture of (2) and (4) with vigorous mixing.

This powder mixture is packed into size 3 hard gelatine capsules in a capsule filling machine.

### Example 12

Capsules containing 350 mg of active substance

| Composition: | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

### Preparation:

(1) is triturated with (3). This trituration is added to the mixture of (2) and (4) with vigorous mixing.

This powder mixture is packed into size 0 hard gelatine capsules in a capsule filling machine.

### Example 13

Suppositories containing 100 mg of active substance

| 1 suppository contains: | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

### Example 14

| | Percentage composition | | | | | |
|---|---|---|---|---|---|---|
| | | | Active | | per | per |
| | | Separating | substance | | capsule | capsule |
| | Core material | layer | layer | Total | [mg] | [mg] |
| Tartaric acid | 61.3 | — | — | 61.3 | 176.7 | 353.4 |
| Gum arabic | 3.1 | 2.8 | | 5.9 | 17.0 | 34.0 |
| Talc | — | 5.6 | 3.2 | 8.8 | 25.4 | 50.7 |
| Hydroxyhydroxypropyl- | — | — | 4.0 | 4.0 | 11.5 | 23.1 |
| cellulose | | | | | | |
| Active substance | — | — | 20.0 | 20.0 | 50.0 | 100.0 |
| (based on the base) | | | | | | |
| Total | | | | 100.0 | 288.3 | 576.5 |

### Example 15

| | Percentage composition | | | | | |
|---|---|---|---|---|---|---|
| | | | Active | | per | per |
| | | Separating | substance | | capsule | capsule |
| | Core material | layer | layer | Total | [mg] | [mg] |
| Tartaric acid | 38.5 | — | — | 38.5 | 55.5 | 166.5 |
| Gum arabic | 1.9 | 1.7 | | 3.6 | 5.2 | 15.6 |
| Talc | — | 3.5 | 6.4 | 9.9 | 14.3 | 42.8 |
| Hydroxyhydroxypropyl- | — | — | 8.0 | 8.0 | 11.5 | 34.6 |
| cellulose | | | | | | |
| Active substance | — | — | 40.0 | 40.0 | 50.0 | 150.0 |
| (based on the base) | | | | | | |
| Total | | | | 100.0 | 144.2 | 432.5 |

The preparation and the structure of the pellets according to Examples 14 and 15 is described in detail in WO 03/074056.

We claim:

1. A method for treatment and/or prophylaxis of a disease selected from the group consisting of:
(a) neurodegenerative disease;
(b) brain micro vessel disease;
(c) diseases which are mediated via PAR 1 to PAR 4 receptors;
(d) oxidative stress induced by thrombin;
(e) haematological diseases;
(f) heparin induced thrombocythompenia;
(g) cancer disease;
(h) thrombosis in polychemotherapy;
(i) central vein thrombosis (CVT);
(j) HIV encephalitis;
(k) rheumatoid disorders;
(l) Tinnitus Aurium and
(m) kidney disease,
comprising the step of administering to a patient in need thereof a therapeutically effective amount of a compound, optionally in the form of tautomers, racemates, enantiomers, diastereomers, pharmacologically acceptable acid addition salts, solvates, hydrates or prodrugs thereof, selected from the group consisting of dabigatran, dabigatran etexilate, 1-methyl-2-[4-(N-hydroxyamidino)-phenylamino-methyl]benzimidazol-5-yl-carboxylic acid-(N-2-pyridyl-N-2-ethoxycarbonylethyl)-amide, melagatran (inogatran), ximelagatran, hirudin, hirolog and argatroban.

2. The method according to claim 1, wherein the neurodegenerative disease is Alzheimer disease.

3. The method according to claim 1, wherein the cancer disease is lung cancer or pancreatic cancer.

4. The method according to claim 1, wherein the rheumatoid disorder is selected from the group consisting of rheumatoid arthritis and systemic lupus erythematodes (SLE).

5. The method according to claim 1, wherein the kidney disease is proteinuria (urinary albumin excretion) in patients with chronic kidney disease or proteinuria (urinary albumin excretion) in patients with Diabetes and albuminuria.

6. The method according to claim 1, wherein the disease is associated with VTE.

7. The method according to claim 1, wherein the compound is selected from the group consisting of dabigatran, dabigatran etexilate and 1-methyl-2-[4-(N-hydroxyamidino)-phenylaminomethyl]-benzimidazol-5-yl-carboxylic acid-(N-2-pyridyl-N-2-ethoxycarbonylethyl)-amide.

8. The method according to claim 1, wherein the compound is selected from the group consisting of dabigatran and dabigatran etexilate or a pharmacologically acceptable acid addition salt thereof.

9. The method according to claim 1, wherein the compound is dabigatran etexilate or a pharmacologically acceptable acid addition salt thereof.

10. The method according to claim 1, wherein the compound is the acid addition salt of dabigatran etexilate with methanesulfonic acid.

11. The method according to claim 1, wherein the compound is applied in a dose range between 0.1 mg to 600 mg per day.
